(19) United States
(12) Patent Application Publication
(10) Pub. No.: US 2010/0221337 A1
(43) Pub. Date: Sep. 2, 2010

(54) METHOD OF TREATING ARTHRITIS, PAIN OR INFLAMMATION WITH NAPROXEN 2(METHANESULFONYL)ETHYL ESTER AND AN H2 RECEPTOR ANTAGONIST

(75) Inventors: Mitchell P. Fink, Pittsburgh, PA (US); Kathryn Lea Sewell, Brighton, MA (US)

Correspondence Address: PEPPER HAMILTON LLP, ONE MELLON CENTER, 50TH FLOOR, 500 GRANT STREET, PITTSBURGH, PA, 15219, US

(73) Assignee: LOGICAL THERAPEUTICS, INC., Waltham, MA (US)

(21) Appl. No.: 12/527,011

(22) PCT Filed: Feb. 14, 2008
(86) PCT No.: PCT/US08/53938

§ 371 Date: May 14, 2010

Related U.S. Application Data
(60) Provisional application No. 60/889,777, filed on Feb. 14, 2007.

(51) Int. Cl. A61K 9/28; A61P 19/02; A61P 29/00; A61K 31/4164; A61K 31/341; A61K 31/426

(52) U.S. Cl. 424/474; 514/400; 514/471; 514/370; 514/365

(57) ABSTRACT

Embodiments of the present invention provide methods of treating pain, arthritis and inflammation comprising administering naproxen 2(methanesulfonyl)ethyl ester and an H2 receptor antagonist. Further embodiments provide pharmaceutical compositions comprising naproxen 2(methanesulfonyl)ethyl ester and an H2 receptor antagonist.

### CROSS-REFERENCE TO RELATED APPLICATIONS

This application claims the benefit of U.S. Provisional Application No. 60/889,777, filed Feb. 14, 2007, which is herein incorporated by reference in its entirety.

### GOVERNMENT INTERESTS

Not applicable

### PARTIES TO A JOINT RESEARCH AGREEMENT

Not applicable

### INCORPORATION BY REFERENCE OF MATERIAL SUBMITTED ON A COMPACT DISC

Not applicable

### BACKGROUND

1. Field of Invention

Not applicable

2. Description of Related Art

Despite the advent of modem pharmaceutical technology, many drugs still possess untoward toxicities which often limit the therapeutic potential thereof. For example, although nonsteroidal anti-inflammatory drugs (NSAIDs) are a class of compounds which are widely used for the treatment of inflammation, pain and fever, NSAIDs (e.g., naproxen, aspirin, ibuprofen and ketoprofen) can cause gastrointestinal ulcers, a side effect that remains the major limitation to the use of NSAIDs.

There are two major ulcerogenic effects of NSAIDs: (1) irritant effects on the epithelium of the gastrointestinal tract and (2) suppression of gastrointestinal prostaglandin synthesis. In recent years, numerous strategies have been attempted to design and develop new NSAIDs that reduce the damage to the gastrointestinal tract. These efforts, however, have not fully satisfied the medical need. For example, enteric coating or slow release formulations designed to reduce the topical irritant properties of NSAIDs have been shown to be ineffective in terms of reducing the incidence of clinically significant side effects, including perforation and bleeding.

It is well recognized that aspirin and other NSAIDs exert their pharmacological effects through the non-selective inhibition of cyclooxygenase (COX) enzymes, thereby blocking prostaglandin synthesis. There are two types of COX enzymes, namely COX1 and COX2. COX1 is expressed constitutively in many tissues, including the stomach, kidney, and platelets, whereas COX2 is expressed only at the she of inflammation. The prostagladins derived from COX1 are responsible for many of the physiological effects, including maintenance of gastric mucosal integrity. Many attempts have been made to develop NSAIDs that only inhibit COX2, without impacting the activity of COX1. There are several NSAIDs (e.g., rofecoxib and celecoxib) that show marked selectivity for COX2. These drugs appear to have reduced gastrointestinal toxicity relative to other NSAIDs. However, the physiological functions of COX1 and COX2 are not always well defined. Thus, there is a possibility that prostaglandins produced as a result of COX1 expression may also contribute to inflammation, pain and fever. On the other hand, prostaglandins produced by COX2 have been shown to play important physiological functions, including the initiation and maintenance of labor and in the regulation of bone resorption, thus inhibition of this pathway may not always be beneficial. Considering these points, highly selective COX2 inhibitors have been known to product cardiovascular side effects and may produce additional side effects above and beyond those observed with standard NSAIDs, therefore such inhibitors may not be highly desirable.

In general, various acid inhibitors may be useful during administration of NSAIDs. For example, more potent and longer lasting acid inhibitors, such as proton pump inhibitors, and shorter acting agents, e.g., histamine H₂ receptor antagonists (H-2 blockers) are two classes of acid inhibitors, with different effects. Gastric pH fluctuates widely throughout the dosing interval with short acting acid inhibitors leaving the mucosa vulnerable for significant periods of time. In particular, the pH is at its lowest point, and hence the mucosa is most vulnerable, at the end of the dosing interval (least amount of acid inhibition) and for some time after the subsequent dose of acid inhibitor. In general, it appears that when a short acting acid inhibitor and an NSAID are administered simultaneously, NSAID-related mucosal damage occurs both before the pH of the gastrointestinal tract can be raised and after the acid inhibiting effect of the short acting acid inhibitor dissipates.

Longer lasting agents, such as proton pump inhibitors (PPIs), usually maintain a consistently higher gastroduodenal pH throughout the day, after several days dosing, their antisecretory effect may be delayed for several hours and may not take full effect for several days. Their effect may be diminished toward the end of the usual dosing interval. Intragastric pH rises particularly slowly with the first does in a course of treatment since this class of drugs is enteric coated to avoid destruction by stomach acid. As a result, absorption is delayed for several hours. Even then, some patients fail to respond consistently to drugs of this type and suffer from “acid breakthrough” which again leaves them vulnerable to NSAID-associated gastroduodenal damage. Despite a significant reduction in gastroduodenal lesions with the concomitant administration of a proton pump inhibitor during six months of NSAID therapy, some patients still develop ulcers, indicating that there remains substantial room for improvement.

Overall, it may be concluded that the risk of inducing GI ulcers is a recognized problem associated with the administration of NSAIDs and that, despite considerable effort, an ideal solution has not yet been found. Accordingly, there is still a need in the art for products which contain an NSAID therapeutic benefit, but which cause a reduced incidence of side-effects.

### BRIEF SUMMARY OF THE INVENTION

Embodiments of the present invention provide methods of treating arthritis, inflammation or pain comprising administering a patient naproxen 2(methanesulfonyl)ethyl ester and an H2 receptor antagonist.

Further embodiments of the present invention provide pharmaceutical compositions comprising a therapeutically effective amount of naproxen 2(methanesulfonyl)ethyl ester, a therapeutically effective amount of an H2 receptor antagonist and one or more excipients.

Further embodiments of the present invention provide methods of treating inflammation or pain in a patient who has had a previous ulcer or gastrointestinal bleeding comprising administering said patient naproxen 2(methanesulfonyl)ethyl ester and an H2 receptor antagonist.

Another embodiment of the present invention provides methods of treating inflammation or pain in a patient who has a factor for a high risk gastrointestinal complication comprising administering said patient naproxen 2(methanesulfonyl)ethyl ester and an H2 receptor antagonist.

Another embodiment of the present invention provides pharmaceutical formulations comprising naproxen 2(methanesulfonyl)ethyl ester and an H2 receptor antagonist, wherein said H2 receptor antagonist is separated from the 2(methanesulfonyl)ethyl ester by a layer of one or more excipients and wherein said pharmaceutical formulation is a tablet.

### DESCRIPTION OF DRAWINGS

Not applicable

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

It must also be noted that as used herein and in the appended claims, the singular forms “a”, “an”, and “the” include plural reference unless the context clearly dictates otherwise. Thus, for example, reference to a “cell” is a reference to one or more cells and equivalents thereof known to those skilled in the art, and so forth.

As used herein, the term “about” means plus or minus 10% of the numerical value of the number with which it is being used. Therefore, about 50% means in the range of 45%-55%.

A “therapeutically effective amount” or “effective amount” of a composition is a predetermined amount calculated to achieve the desired effect. The activity contemplated by the present methods includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, and the condition being treated. It will be understood that the effective amount administered will be determined by the physician in the light of the relevant circumstances including the condition to be treated, the choice of compound to be administered, and the chosen route of administration, and therefore the above dosage ranges are not intended to limit the scope of the invention in any way. A therapeutically effective amount of compound of this invention is typically an amount such that when it is administered in a physiologically tolerable excipient composition, it is sufficient to achieve an effective systemic concentration or local concentration in the tissue.

The terms “treat”, “treated,” or “treating” as used herein refers to both therapeutic treatment and prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

Optical Isomers—Diastereorners—Geometric Isomers—Tautomers. Compounds described herein may contain an asymmetric center and may thus exist as enantiomers. Where the compounds according to the invention possess two or more asymmetric centers, they may additionally exist as diastereomers. The present invention includes all such possible stereoisomers as substantially pure resolved enantiomers, racemic mixtures thereof, as well as mixtures of diastereomers. The formulas are shown without a definitive stereochemistry at certain positions. The present invention includes all stereoisomers of such formulas and pharmaceutically acceptable salts thereof. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general formula may be obtained by stereospecific synthesis using optically pure starting materials or reagents of known configuration.

The present invention is based upon the discovery of improved methods of treatment and pharmaceutical compositions for administering naproxen 2(methanesulfonyl)ethyl ester to patients. In addition to containing naproxen 2(methanesulfonyl)ethyl ester, the compositions include H² receptor antagonists that are capable of raising the pH of the GI tract of patients. In particular, patients in need of treatment for arthritis, inflammation and pain can benefit from this invention.

In one embodiment, the invention comprises a method of treating arthritis, inflammation or pain comprising administering a patient naproxen 2(methanesulfonyl)ethyl ester and an H2 receptor antagonist. In another embodiment, the invention comprises a method of treating arthritis, inflammation or pain in a patient at risk for having an ulcer a comprising administering naproxen 2(methanesulfonyl)ethyl ester and an H2 receptor antagonist. In a further embodiment, the invention comprises a method of treating arthritis, inflammation or pain in a patient who has had a previous ulcer or gastrointestinal bleeding a comprising administering naproxen 2(methanesulfonyl)ethyl ester and an H2 receptor antagonist.

Examples of H2 receptor antagonist a useful for this invention include, but are not limited to, cimetidine, famotidine, nizatidine, and ranitidine. Included within these examples are salts, isomers, racemic compounds, crystals, polymorphs, amorphous forms and cocrystals of these examples.

In a still further embodiment, the invention comprises a method of treating arthritis, inflammation or pain in a patient who has a high risk factor for receiving a gastrointestinal disorder comprising administering naproxen 2(methanesulfonyl)ethyl ester and an H2 receptor antagonist. Patients who have high risk factors for receiving a gastrointestinal disorder include patients of age over 60 years, patients taking aspirin therapy, patients taking corticosteroids and patients who have had a previous ulcer or gastrointestinal bleeding event.

In one embodiment, the invention comprises a medicament for the treatment of arthritis, inflammation or pain comprising naproxen 2(methanesulfonyl)ethyl ester and an H2 receptor antagonist. In another embodiment, the invention comprises a medicament for treatment of arthritis, inflammation or pain in a patient who has had a previous ulcer or gastrointestinal bleeding comprising naproxen 2(methanesulfonyl)ethyl ester and an H2 receptor antagonist. In another embodiment, the invention comprises a medicament for treatment of arthritis, inflammation or pain in a patient who has a high risk factor for receiving a gastrointestinal disorder comprising naproxen 2(methanesulfonyl)ethyl ester and an H2 receptor antagonist. Patients who have high risk factors for receiving a gastrointestinal disorder include patients of age over 60 years, patients taking aspirin therapy, and patients taking corticosteroids.

Included within the definition of arthritis, but not limited to, is rheumatoid arthritis, osteoarthritis, juvenile rheumatoid arthritis, ankylosing spondosis, juvenile arthritis, bursitis, gout, Psoriatic arthritis, and Reactive arthritis as described at http://www.arthritis.org/disease-center.php?disease_id=3.

In one embodiment, the invention comprises a pharmaceutical composition of naproxen 2(methanesulfonyl)ethyl ester and an H2 receptor antagonist. In another embodiment, the invention comprises a pharmaceutical composition of naproxen 2(methanesulfonyl)ethyl ester and cimetidine or a pharmaceutically acceptable salt thereof. In a further embodiment, the invention comprises a pharmaceutical composition of naproxen 2(methanesulfonyl)ethyl ester and famotidine or a pharmaceutically acceptable salt thereof. In still further embodiment, the invention comprises a pharmaceutical composition of naproxen 2(methanesulfonyl)ethyl ester and nizatidine or a pharmaceutically acceptable salt thereof. In another embodiment, the invention comprises a pharmaceutical composition of naproxen 2(methanesulfonyl)ethyl ester and ranitidine or a pharmaceutically acceptable salt thereof. In one embodiment, the invention comprises a pharmaceutical composition of naproxen 2(methanesulfonyl)ethyl ester and ranitidine bismuth citrate or a pharmaceutically acceptable salt thereof.

For some patients the combination of the two drugs might be more useful co-packaged as opposed to combined in the same pill or tablet. In another embodiment, the invention comprises a package comprising naproxen 2(methanesulfonyl)ethyl ester and said H2 receptor antagonist. In another embodiment, the invention comprises a package of naproxen 2(methanesulfonyl)ethyl ester and cimetidine or a pharmaceutically acceptable salt thereof. In a further embodiment, the invention comprises a package of naproxen 2(methanesulfonyl)ethyl ester and famotidine or a pharmaceutically acceptable salt thereof. In still further embodiment, the invention comprises a package of naproxen 2(methanesulfonyl)ethyl ester and nizatidine or a pharmaceutically acceptable salt thereof. In another embodiment, the invention comprises a package of naproxen 2(methanesulfonyl)ethyl ester and ranitidine or a pharmaceutically acceptable salt thereof. In one embodiment, the invention comprises a package of naproxen 2(methanesulfonyl)ethyl ester and ranitidine bismuth citrate or a pharmaceutically acceptable salt thereof.

In another embodiment, the invention comprises a pharmaceutical formulation comprising naproxen 2(methanesulfonyl)ethyl ester and an H2 receptor antagonist wherein said H2 receptor antagonist is separated from the naproxen 2(methanesulfonyl)ethyl ester by a layer of one or more excipients and wherein said pharmaceutical formulation is a tablet. In a further embodiment, the invention comprises a pharmaceutical formulation comprising naproxen 2(methanesulfonyl)ethyl ester and an H2 receptor antagonist wherein said H2 receptor antagonist is separated from the naproxen 2(methanesulfonyl)ethyl ester by a layer of one or more excipients and wherein said pharmaceutical formulation is an enteric coated tablet.

Compositions of this invention can be used to treat arthritis, pain and inflammation while also reducing the patient's likelihood of having a duodenal ulcer, a gastric ulcer, gastroesophageal reflux disease, gastrointestinal bleeding or erosive esophagitis.

It is expected that a skilled pharmacologist may adjust the amount of drug in a pharmaceutical composition or administered to a patient based upon standard techniques well known in the art. With respect to a H2 receptor antagonists, tablets or capsules may contain anywhere from 1 mg to 1000 mg per unit dose. For example, the H2 receptor antagonist cimetidine may be present in tablets or capsules in an amount from 50 to 1000 mg. Other typical amounts are: ranitidine, 50-200 mg famotidine, 5-50 nizatidine, 50-400 mg.

Naproxen 2(methanesulfonyl)ethyl ester is disclosed in U.S. Pat. No. 6,355,666 (application Ser. No. 09/602,688), herein incorporated by reference in its entirety, as Compound 50 and a method of making Compound 50 is disclosed in Example 17. Naproxen 2(methanesulfonyl)ethyl ester is also called (S)-(+)-2-(6-methoxy-2-naphthyl)propionic acid 2-methanesulfonyl ethyl ester. The structure of naproxen 2(methanesulfonyl)ethyl ester is

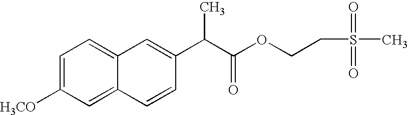

In certain embodiments, the pharmaceutical compositions of the invention include tablets, dragees, liquids and capsules and can he made in accordance with methods that are standard in the art (see, e.g. Remington's Pharmaceutical Sciences, 16th ea., A Oslo editor, Easton, Pa. (1980)). Drugs and drug combinations will typically be prepared in admixture with conventional excipients.

Enteric coating layer(s) may be applied onto a tablet using standard coating techniques. The enteric coating materials may be dissolved or dispersed in organic or aqueous solvents and may include one or more of the following materials: methacrylic acid copolymers, shellac, hydroxypropylmethcellulose phthalate, polyvinyl acetate phthalate, hydroxypropylmethylcellulose trimellitate, carboxymethylethyl-cellulose, cellulose acetate phthalate or other suitable enteric coating polymer(s). The pH at which the enteric coat will dissolve can be controlled by the polymer or combination of polymers selected and/or ratio of pendant groups. For example, dissolution characteristics of the polymer film can be altered by the ratio of free carboxyl groups to ester groups. Enteric coating layers also contain pharmaceutically acceptable plasticizers such as triethyl citrate, dibutyl phthalate, triacetin, polyethylene glycols, polysorbates or other plasticizers. Additives such as dispersants, colorants, anti-adhering and anti-foaming agents may also be included.

In one embodiment, the combination of an H2 receptor antagonist and naproxen 2(methanesulfonyl)ethyl ester will be in the form of a bi- or multi-layer tablet. In a bilayer configuration, one portion of the tablet contains the H2 receptor antagonist in the required dose along with the appropriate excipients, agents to aid dissolution, lubricants, fillers, etc. The second portion of the tablet will contain naproxen 2(methanesulfonyl)ethyl ester, in the required dose along with other excipients, dissolution agents, lubricants, fillers, etc. In one exemplary embodiment, the naproxen 2(methanesulfonyl)ethyl ester layer is surrounded by a polymeric coating which does not dissolve at a pH of less than 4. The naproxen 2(methanesulfonyl)ethyl ester may be granulated by methods such as slugging, low- or high-shear granulation, wet granulation, or fluidized-bed granulation. Of these processes, slugging generally produced tablets of less hardness and greater friability. Low-shear granulation, high shear granulation, wet granulation and fluidized-bed granulation generally produce harder, less friable tablets.

Specific modes of administration will depend on the indication. The selection of the specific route of administration and the dose regimen is to be adjusted or titrated by the clinician according to methods known to the clinician in order to obtain the optimal clinical response. The amount of compound to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the e.g., by the clinician),

Pharmaceutical formulations containing the compounds of the present invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present invention. It is also known in the an that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compounds of the present invention can be formulated for parenteral administration by injection, e.g., by bolus injection or continuous infusion. The compounds can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compounds can be formulated readily by combining these compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone. If desired, disintegrating agents can be added, such as, but not limited to, the cross-linked polyvinyl pyrrolidone, agar, or alginic acid or a salt thereof such as sodium alginate.

Dragee cores can he provided with suitable coatings. For this purpose, concentrated sugar solutions can be used, which can optionally contain gum arabic, talc, polyvinyl pyrrolidone, caxbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of e.g., tablets or lozenges formulated in a conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds of the present invention can also be formulated in rectal compositions such as suppositories or retention enemas, e.g., containing conventional suppository bases such as cocoa butter or other glycerides.

In addition to the formulations described previously, the compounds of the present invention can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compounds of the present invention, for example, can be applied to a plaster, or can be applied by transdermal, therapeutic systems that are consequently supplied to the organism.

Pharmaceutical compositions of the compounds also can comprise suitable solid or gel phase carriers or excipients. Examples of such colliers or excipients include but are not limited to calcium carbonate, calcium phosphate, various sugars, starches, cellulose derivatives, gelatin, and polymers such as, e.g., polyethylene glycols,

The compounds of the present invention can also be administered in combination with other active ingredients, such as, for example, adjuvants, protease inhibitors, or other compatible drugs or compounds where such combination is seen to be desirable or advantageous in achieving the desired effects of the methods described herein. It is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. Nothing herein is to be construed as an admission that the invention is not entitled to antedate such disclosure by virtue of prior invention.

Although the present invention has been described in considerable detail with reference to certain preferred embodiments thereof, other versions are possible. Therefore the spirit and scope of the appended claims should not be limited to the description and the preferred versions contained within this specification.

### Example 1

The following compositions are representative compositions which could be made according to this invention.

A. Naproxen 2(methanesulfonyl)ethyl ester and 100 mg cimetidine

B. Naproxen 2(methanesulfonyl)ethyl ester and 200 mg cimetidine

C. Naproxen 2(methanesulfonyl)ethyl ester and 300 mg cimetidine

D. Naproxen 2(methanesulfonyl)ethyl ester and 400 mg cimetidine

E. Naproxen 2(methanesulfonyl)ethyl ester and 800 trig cimetidine

F. Naproxen 2(methanesulfonyl)ethyl ester and 300 mg cimetidine hydrochloride

G. Naproxen 2(methanesulfonyl)ethyl ester and 75 mg ranitidine hydrochloride

H. Naproxen 2(methanesulfonyl)ethyl ester and 150 mg ranitidine hydrochloride

I. Naproxen 2(methanesulfonyl)ethyl ester and 10 mg famotidine

J. Naproxen 2(methanesulfonyl)ethyl ester and 20 mg famotidine

K. Naproxen 2(methanesulfonyl)ethyl ester and 40 mg famotidine

L. Naproxen 2(methanesulfonyl)ethyl ester and 75 mg nizatidine

M. Naproxen 2(methanesulfonyl)ethyl ester and 150 mg nizatidine

N. Naproxen 2(methanesulfonyl)ethyl ester and 300 mg nizatidine

Any one of the above compositions could be combined with one or more excipients.

What is claimed is:

1. A method of treating arthritis, inflammation or pain comprising administering a patient naproxen 2(methanesulfonyl)ethyl ester and an 1-12 receptor antagonist.

2. The method of claim 1, wherein said H2 receptor antagonist is selected from cimetidine, ranitidine, famotidine, and nizatidine.

3. The method of claim 1, wherein said patient is a patient at risk for having an ulcer.

4. The method of claim 1, wherein said naproxen 2(methanesulfonyl)ethyl ester and said H2 receptor antagonist are co-packaged together.

5. The method of claim 1, wherein said naproxen 2(methanesulfonyl)ethyl ester and said H2 receptor antagonist are present in the same pharmaceutical composition.

6. The method of claim 5, wherein said pharmaceutical composition is a tablet.

7. The method of claim 1, wherein said arthritis is selected from the group consisting of rheumatoid arthritis and osteoarthritis.

8. A pharmaceutical composition comprising a therapeutically effective amount of naproxen 2(methanesulfonyl)ethyl ester, a therapeutically effective amount of an H2 receptor antagonist and one or more excipients.

9. The pharmaceutical composition of claim 8, wherein said H2 receptor antagonist is selected from cimetidine, ranitidine, famotidine, and nizatidine.

10. The pharmaceutical composition of claim 8, wherein said pharmaceutical composition is a tablet.

11. A method of treating inflammation or pain in a patient who has had a previous ulcer or gastrointestinal bleeding comprising administering said patient naproxen 2(methanesulfonyl)ethyl ester and an H2 receptor antagonist.

12. The method of claim 11, wherein said H2 receptor antagonist is selected from cimetidine, ranitidine, famotidine, and nizatidine.

13. The method of claim 11, wherein said patient is a patient at risk for having an ulcer.

14. The method of claim 11, wherein said naproxen 2(methanesulfonyl)ethyl ester and said H2 receptor antagonist are co-packaged together.

15. The method of claim 11, wherein said naproxen 2(methanesulfonyl)ethyl ester and said H2 receptor antagonist are present in the same pharmaceutical composition.

16. The method of claim 15, wherein said pharmaceutical composition is a tablet.

17. A method of treating inflammation or pain in a patient who has a factor for a high risk gastrointestinal complication comprising administering said patient naproxen 2(methanesulfonyl)ethyl ester and an H2 receptor antagonist.

18. The method of claim 17, wherein said factor is an age of 60 or more years.

19. The method of claim 17, wherein said factor is concurrent treatment with aspirin or a corticosteroid.

20. A pharmaceutical formulation comprising naproxen 2(methanesulfonyl)ethyl ester and an H2 receptor antagonist, wherein said H2 receptor antagonist is separated from the 2(methanesulfonyl)ethyl ester by a layer of one or more excipients and wherein said pharmaceutical formulation is a tablet.

21. The tablet of claim 20, wherein said tablet is covered by an enteric coating.
